SEQUENCE LISTING (18 sequences)

SEQ ID NO: 1 - DNA, 1623 bp, Escherichia coli
  CDS (1)..(1623)
atgcgcgttaacaatggtttgaccccgcaagaactcgaggcttatggtatcagtgacgtacatgatatcgtttacaacccaagctacgacctgctgtatcaggaagagctcgatccgagcctgacaggttatgagcgcggggtgttaactaatctgggtgccgttgccgtcgataccgggatcttcaccggtcgttcaccaaaagataagtatatcgtccgtgacgataccactcgcgatactttctggtgggcagacaaaggcaaaggtaagaacgacaacaaacctctctctccggaaacctggcagcatctgaaaggcctggtgaccaggcagctttccggcaaacgtctgttcgttgtcgacgctttctgtggtgcgaacccggatactcgtctttccgtccgtttcatcaccgaagtggcctggcaggcgcattttgtcaaaaacatgtttattcgcccgagcgatgaagaactggcaggtttcaaaccagactttatcgttatgaacggcgcgaagtgcactaacccgcagtggaaagaacagggtctcaactccgaaaacttcgtggcgtttaacctgaccgagcgcatgcagctgattggcggcacctggtacggcggcgaaatgaagaaagggatgttctcgatgatgaactacctgctgccgctgaaaggtatcgcttctatgcactgctccgccaacgttggtgagaaaggcgatgttgcggtgttcttcggcctttccggcaccggtaaaaccaccctttccaccgacccgaaacgtcgcctgattggcgatgacgaacacggctgggacgatgacggcgtgtttaacttcgaaggcggctgctacgcaaaaactatcaagctgtcgaaagaagcggaacctgaaatctacaacgctatccgtcgtgatgcgttgctggaaaacgtcaccgtgcgtgaagatggcactatcgactttgatgatggttcaaaaaccgagaacacccgcgtttcttatccgatctatcacatcgataacattgttaagccggtttccaaagcgggccacgcgactaaggttatcttcctgactgctgatgctttcggcgtgttgccgccggtttctcgcctgactgccgatcaaacccagtatcacttcctctctggcttcaccgccaaactggccggtactgagcgtggcatcaccgaaccgacgccaaccttctccgcttgcttcggcgcggcattcctgtcgctgcacccgactcagtacgcagaagtgctggtgaaacgtatgcaggcggcgggcgcgcaggcttatctggttaacactggctggaacggcactggcaaacgtatctcgattaaagatacccgcgccattatcgacgccatcctcaacggttcgctggataatgcagaaaccttcactctgccgatgtttaacctggcgatcccaaccgaactgccgggcgtagacacgaagattctcgatccgcgtaacacctacgcttctccggaacagtggcaggaaaaagccgaaaccctggcgaaactgtttatcgacaacttcgataaatacaccgacacccctgcgggtgccgcgctggtagcggctggtccgaaactgtaa

SEQ ID NO: 2 - protein, 540 aa, Escherichia coli
MRVNNGLTPQELEAYGISDVHDIVYNPSYDLLYQEELDPSLTGYERGVLTNLGAVAVDTGIFTGRSPKDKYIVRDDTTRDTFWWADKGKGKNDNKPLSPETWQHLKGLVTRQLSGKRLFVVDAFCGANPDTRLSVRFITEVAWQAHFVKNMFIRPSDEELAGFKPDFIVMNGAKCTNPQWKEQGLNSENFVAFNLTERMQLIGGTWYGGEMKKGMFSMMNYLLPLKGIASMHCSANVGEKGDVAVFFGLSGTGKTTLSTDPKRRLIGDDEHGWDDDGVFNFEGGCYAKTIKLSKEAEPEIYNAIRRDALLENVTVREDGTIDFDDGSKTENTRVSYPIYHIDNIVKPVSKAGHATKVIFLTADAFGVLPPVSRLTADQTQYHFLSGFTAKLAGTERGITEPTPTFSACFGAAFLSLHPTQYAEVLVKRMQAAGAQAYLVNTGWNGTGKRISIKDTRAIIDAILNGSLDNAETFTLPMFNLAIPTELPGVDTKILDPRNTYASPEQWQEKAETLAKLFIDNFDKYTDTPAGAALVAAGPKL

SEQ ID NO: 3 - DNA, 36 bp, Artificial Sequence
  Description of Artificial Sequence primer
caatctagaaagacaggattgggtaaatggcaaagg

SEQ ID NO: 4 - DNA, 34 bp, Artificial Sequence
  Description of Artificial Sequence primer
ccgttggtcgactacagcagacgggcgcgaatgg

SEQ ID NO: 5 - DNA, 35 bp, Artificial Sequence
  Description of Artificial Sequence primer
tacattgtcgacatgaaactggatgaaatcgctcg

SEQ ID NO: 6 - DNA, 32 bp, Artificial Sequence
  Description of Artificial Sequence primer
tttgttgtcgaccttagctacggctgagcacg

SEQ ID NO: 7 - DNA, 25 bp, Artificial Sequence
  Description of Artificial Sequence primer
tgatcggatccgaaatgagatataa

SEQ ID NO: 8 - DNA, 24 bp, Artificial Sequence
  Description of Artificial Sequence primer
ctgcggtcgacgtccattgctttc

SEQ ID NO: 9 - DNA, 29 bp, Artificial Sequence
  Description of Artificial Sequence primer
gaccatagatctgaattcgagctcggtac

SEQ ID NO: 10 - DNA, 29 bp, Artificial Sequence
  Description of Artificial Sequence primer
acggccagatctaagcttgcatgcctgca

SEQ ID NO: 11 - DNA, 34 bp, Artificial Sequence
  Description of Artificial Sequence primer
aacagtgatcatttgcctggcggcagtagcgcgg

SEQ ID NO: 12 - DNA, 41 bp, Artificial Sequence
  Description of Artificial Sequence primer
ataaaaagcttagatctcaaaaagagtttgtagaaacgcaa

SEQ ID NO: 13 - DNA, 39 bp, Artificial Sequence
  Description of Artificial Sequence primer
cgtttaagatcttggtttatgtggaaattgtcgaagaga

SEQ ID NO: 14 - DNA, 74 bp, Artificial Sequence
  Description of Artificial Sequence primer
gagttcttgcggggtcaaaccattgttaacgcgcattatatctccttcgagaaacacatttctgcgcatttatc

SEQ ID NO: 15 - DNA, 34 bp, Artificial Sequence
  Description of Artificial Sequence primer
tagcgaagatctctgatgtccggcggtgcttttg

SEQ ID NO: 16 - DNA, 57 bp, Artificial Sequence
  Description of Artificial Sequence primer
ttcagaaaataatcaaaaaaagttagcgtggtgaatttacgccccgccctgccactc

SEQ ID NO: 17 - DNA, 25 bp, Artificial Sequence
  Description of Artificial Sequence primer
gcacttcgcgccgttcataacgata

SEQ ID NO: 18 - DNA, 1188 bp, Artificial Sequence
  Description of Artificial Sequence Cm- resistance gene, PppsA promoter and SD sequence of gene 10 from phage T7
ttacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagagatcttggtttatgtggaaattgtcgaagagagcagatttgcgcaacgctgggatcagtcttaaaaagtaaaaaaatatatttgcttgaacgattcaccgtttttttcatccggttaaatatgcaaagataaatgcgcagaaatgtgtttctcgaaggagatata

(19) United States
(12) Patent Application Publication
(10) Pub. No.: US 2006/0035348 A1
(43) Pub. Date: Feb. 16, 2006

(54) Method for producing an L-amino acid using a bacterium having enhanced expression of the pckA gene

(76) Inventors: Andrey Yurievich Gulevich, Moscow (RU); Irina Vladimirovna Biryukova, Moscow (RU); Danila Vadimovich Zimenkov, Moscow (RU); Aleksandra Yurievna Skorokhodova, Moscow (RU); Aleksandr Dmitrievich Kivero, Moscow (RU); Alla Valentinovna Belareva, Moscow (RU); Sergei Vladimirovich Mashko, Moscow (RU)

Correspondence Address: CERMAK & KENEALY LLP;ACS LLC, 515 EAST BRADDOCK ROAD, SUITE B, ALEXANDRIA, VA, 22314, US

(21) Appl. No.: 11/235,254

(22) Filed: Sep. 27, 2005

Related U.S. Application Data
(63) Continuation of application No. PCT/JP04/04968, filed on Apr. 6, 2004.

(30) Foreign Application Priority Data
Apr. 7, 2003 (RU) 2003109477

(51) Int. Cl. C12P 13/04; C12N 1/21

(52) U.S. Cl. 435/106; 435/252.33

(57) ABSTRACT

The present invention relates to a method for producing L-amino acids, such as L-tryptophan, L-phenylalanine, and L-tyrosine, using a bacterium belonging to the genus Escherichia, and wherein the L-amino acid productivity of said bacterium is enhanced by enhancing a PEP carboxykinase activity, which is coded by the pckA gene.

This application is a continuation under 35 U.S.C. § 120 of PCT/JP2004/004968, filed Apr. 6, 2004, the entirety of which is incorporated by reference.

### BACKGROUND OF THE INVENTION

1. Technical field

The present invention relates to biotechnology, specifically to a method for producing L-amino acid by fermentation, and more specifically to a gene derived from an Escherichia coli bacterium. The gene is useful for improving production of L-amino acids, namely aromatic L-amino acids, such as L-tryptophan, L-phenylalanine, and L-tyrosine.

2. Background art

Conventionally, L-amino acids have been industrially produced by fermentation methods utilizing microorganism strains obtained from natural sources, or mutants thereof which have been especially modified to enhance L-amino acid production.

Many techniques have been disclosed which describe methods for enhancing L-amino acid production. For example, such techniques include transforming a microorganism with recombinant DNA (see, for example, U.S. Pat. No. 4,278,765). Many of these techniques are based on increasing the activities of enzymes involved in amino acid biosynthesis, and/or desensitizing enzymes which are involved in feedback inhibition of the target L-amino acid (see, for example, Japanese Laid-open application No56-18596 (1981), WO 95/16042 or U.S. Pat. Nos. 5,661,012 and 6,040,160).

Phosphoenolpyruvate (PEP) and erythrose 4-phosphate (E4p) are the essential precursors of the common biosynthetic pathway for aromatic L-amino acids. Also, phosphoenolpyruvate is a key intermediate involved in several other cellular processes. In wild-type E. coli, the major PEP consumer is the phosphotransferase system (PTS). Other PEP-consuming enzymes are phosphoenolpyruvate carboxylase, which is encoded by ppc gene, and pyruvate kinases, which are encoded by the pykA and pykF genes. The reactions which result in PEP formation in E. coli are catalyzed by the glycolytic enzyme enolase and gluconeogenesis enzymes phosphoenolpyruvate synthase and phosphoenolpyruvate carboxykinase (PEP carboxykinase), which are encoded by the eno, ppsA and pckA genes, respectively (Escherichia coli and Salmonella, Second Edition, Editor in Chief: F. C. Neidhardt, ASM Press, Washington D.C., 1996).

Therefore, optimization of the specific pathways of PEP and E4P biosynthesis can improve production of aromatic L-amino acids. In particular, increasing of the PEP supply is a standard method for increasing aromatic L-amino acid production. One way of increasing the PEP supply in a cell is to avoid PEP consumption in the phosphotransferase system by use of a non-PTS carbon source. Another way is to make glucose uptake and phosphorylation PEP-independent. Yet another way to increase the PEP supply is to recycle PEP from its derivatives, such as pyruvate and oxaloacetate. The inactivation of PEP carboxylase or PEP kinases to prevent the PEP utilization in glycolysis can also be utilized. Simultaneous inactivation of both the pykA and pykF genes significantly increases carbon flow from glucose to L-phenylalanine (Grinter, N. J., ChemTech, 1998, 33-37 (July)). On the other hand, significantly increased formation of L-phenylalanine in the L-phenylalanine-producing E. coli strain containing an inactivated ppc gene is accompanied by increased formation of unwanted by-products, such as acetate and pyruvate (Miller J. E. et al., J. Ind. Microbiol., 1987, 2, 143-149).

Conversion of pyruvate back into PEP can be performed by the overexpression of phosphoenolpyruvate synthase, which is encoded by the ppsA gene. In this case, the carbon flux will be successfully directed toward DAHP (3-deoxy-D-arabino-heptulosonate 7-phosphate) production (Patnaik R. and Liao J. C., Appl. Environ. Microbiol., 1994, 60, 3903-3908, U.S. Pat. No. 5,906,925, U.S. Pat. No. 5,985,617, U.S. Pat. No. 6,489,100; PCT application WO9608567A1). It was shown that increasing the phosphoenolpyruvate synthase activity in coryneform bacterium cells leads to increased production of L-amino acids, such as L-lysine, L-glutamic acid, L-threonine, L-isoleucine, and L-serine (PCT application WO0056859A1). Also, it was shown that enhancing activity of phosphoenolpyruvate synthase in coryneform bacterium cells or in bacterium belonging to the genus Escherichia is useful for production of L-tryptophan, L-phenylalanine, L-tyrosine, L-threonine, and L-isoleucine (European patent application EP0877090A1).

It is known that growth in the presence of dicarboxylic acids or acetate, formation of PEP and intermediates of the glycolytic pathway requires, in particular, decarboxylation of oxaloacetate by PEP carboxykinase (PckA), giving PEP in an ATP-dependent reaction. On the other hand, growth in the presence of glucose exhibits the highest expression of the ppc gene and the lowest expression of the pckA gene (Teroaka H. et al., J. Biochem. 1970, 67, 567-575; Goldie H., J. Bacteriol. 1984, 59, 832-838).

Using stoichiometric pathway analysis, a novel pathway to recycle pyruvate to PEP was proposed but not experimentally proven. This hypothetical cycle was considered to consist of PEP carboxykinase (PckA) that converted oxaloacetate to PEP and the glyoxylate shunt (Liao J. C. et al., Biotechnol. Bioeng., 1996, 52, 129-140).

It has previously been shown that attenuation of, or switching off of the pckA gene and/or the yjfA and ytfP open reading frames in the cells of microorganisms of the Enterobacteriaceae family is useful for production of L-threonine (PCT application WO0229080A2). But at present there are no reports describing the fact that enhancement of PEP carboxykinase activity in the cell of L-amino acid-producing bacterium leads to an increase in the L-amino acid production.

### SUMMARY OF THE INVENTION

An object of the present invention is to enhance the productivity of aromatic L-amino acid-producing strains and to provide a method for producing the aromatic L-amino acid using the strain.

It is an object of the present invention to provide an L-amino acid-producing bacterium belonging to the genus Escherichia, wherein the bacterium has been modified to have an enhanced PEP carboxykinase activity.

It is a further object of the present invention to provide the bacterium as described above, wherein the PEP carboxykinase activity is enhanced by modifying an expression control sequence of the PEP carboxykinase gene on the chromosome of the bacterium so that the expression of the gene is enhanced, or by increasing the copy number of the gene.

It is a further object of the present invention to provide the bacterium as described above, wherein the native promoter of said gene is replaced with a more potent promoter.

It is a further object of the present invention to provide the bacterium as described above, wherein the native SD sequence of said gene is replaced with a more efficient SD sequence.

It is a further object of the present invention to provide the bacterium as described above, wherein the PEP carboxykinase gene originates from a bacterium belonging to the genus Escherichia.

It is a further object of the present invention to provide the bacterium as described above, wherein the PEP carboxykinase gene encodes a protein selected from the group consisting of:

(A) a protein comprising the amino acid sequence in SEQ ID NO: 2; and

(B) a protein comprising the amino acid sequence in SEQ ID NO: 2 which includes deletion, substitution, insertion, or addition of one or several amino acids, and which has an activity of PEP carboxykinase. (hereinafter, the proteins as defined in the above (A) or (B) are referred to as “protein(s) of the present invention”)

It is a further object of the present invention to provide the bacterium as described above, wherein the PEP carboxykinase gene comprises a DNA selected from the group consisting of:

(a) a DNA comprising the nucleotide sequence of the nucleotides 1 to 1623 in SEQ ID NO: 1; and

(b) a DNA which is able to hybrid with the nucleotide sequence of the nucleotides 1-1623 in SEQ ID NO: 1, or a probe which can be prepared from the nucleotide sequence under stringent conditions and encodes a protein having a PEP carboxykinase activity.

It is a further object of the present invention to provide the bacterium as described above, wherein the stringent conditions are conditions in which washing is performed at 60° C., and at a salt concentration corresponding to 1×SSC and 0.1% SDS.

It is a further object of the present invention to provide the bacterium as described above, wherein the bacterium is further modified to have enhanced expression of the yddG open reading frame.

It is a further object of the present invention to provide the bacterium as described above, wherein the L-amino acid is an aromatic L-amino acid selected from the group consisting of L-tryptophan, L-phenylalanine, and L-tyrosine.

It is a further object of the present invention to provide a method for producing aromatic L-amino acids, comprising cultivating the bacterium as described above in a culture medium, and collecting the L-amino acid from the culture medium.

It is a further object of the present invention to provide the method as described above, wherein the L-amino acid is an aromatic amino acid selected from the group consisting of L-tryptophan, L-phenylalanine, and L-tyrosine.

It is a further object of the present invention to provide the method as described above, wherein the bacterium has enhanced expression of genes for aromatic amino acid biosynthesis.

The method for producing an L-amino acid includes production of L-tryptophan using L-tryptophan-producing bacterium, wherein the activity of the protein of the present invention is enhanced.

### BRIEF DESCRIPTION OF THE DRAWING

FIG. 1 shows the structure of the constructed chromosome region upstream of the pckA gene.

### DETAILED DESCRIPTION OF THE PREFERRED EMBODIMENTS

The present inventors have found that enhancement of transcription and/or translation of the pckA gene can enhance L-tryptophan production when said gene is expressed in an L-tryptophan-producing strain. The pckA gene encodes PEP carboxykinase, which catalyzes conversion of oxaloacetate to PEP. Thus the present invention has been completed.

The present invention will be explained in detail below.

The bacterium of the present invention is an L-amino acid-producing bacterium belonging to the genus Escherichia that has enhanced activity of a protein, and as a result, enhances production of a target L-amino acid. More specifically, the bacterium of the present invention is an aromatic L-amino acid-producing bacterium belonging to the genus Escherichia that has enhanced activity of the protein of the present invention. Even more specifically, the bacterium of the present invention is an aromatic L-amino acid-producing bacterium, such as an L-tryptophan-producing bacterium, belonging to the genus Escherichia, wherein the bacterium has been modified to have enhanced activity of PEP carboxykinase. More specifically, the bacterium of present invention harbors the DNA comprising the pckA, gene, and has a modified expression control sequence in the chromosome of the bacterium, and has enhanced ability to produce L-tryptophan.

The phrase “L-amino acid-producing bacterium” means a bacterium having an ability to cause accumulation of an L-amino acid in a medium when the bacterium of the present invention is cultured in the medium. The L-amino acid producing ability may be imparted or enhanced by breeding. The phrase “L-amino acid-producing bacterium” as used herein also means a bacterium which is able to produce and cause accumulation of an L-amino acid in a culture medium in an amount larger than a wild-type or parental strain, and preferably means that the microorganism is able to produce and cause accumulation in a medium of an amount not less than 0.5 g/L, more preferably not less than 1.0 g/L of a target L-amino acid. The term “L-amino acid” includes L-alanine, L-arginine, L-asparagine, L-aspartic acid, L-cysteine, L-glutamic acid, L-glutamine, L-glycine, L-histidine, L-isoleucine, L-leucine, L-lysine, L-methionine, L-phenylalanine, L-proline, L-serine, L-threonine, L-tryptophan, L-tyrosine, and L-valine, and preferably includes aromatic L-amino acid, such as L-tryptophan, L-phenylalanine, and L-tyrosine.

The phrase “a bacterium belonging to the genus Escherichia” means that the bacterium is classified as the genus Escherichia according to the classification known to a person skilled in the art of microbiology. Examples of the microorganism belonging to the genus Escherichia used in the present invention include Escherichia coli (E. coli).

The phrase “activity of PEP carboxykinase” means an activity to catalyze the reaction of converting the oxaloacetate into phosphoenolpyruvate, while consuming ATP and releasing ADP and carbon dioxide. Activity of a PEP carboxykinase could be measured by the method described by, for example, Krebs A. and Bridger W. A. (Can. J. Biochem., 1980, 58, 309-318).

The phrase “modified to have enhanced activity of PEP carboxykinase” means that the activity of PEP carboxylase per cell is higher than that of a non-modified strain, for example, a wild-type strain. For example, strains in which the number of PEP carboxykinase molecules per cell is increased, strains in which the specific activity per PEP carboxykinase molecule is increased, and so forth, are encompassed. Furthermore, a wild-type strain that serves as an object for comparison includes, for example, the Escherichia coli K-12. As a result of enhancement of intracellular activity of PEP carboxykinase, an effect is obtained whereby the amount of L-tryptophan which has accumulated in a medium increases.

Enhancement of PEP carboxykinase activity in a bacterial cell can be attained by enhancing expression of a gene coding for PEP carboxykinase. As the PEP carboxykinase gene, any of the genes derived from bacteria belonging to the genus Escherichia, as well as genes derived from other bacteria such as coryneform bacteria, can be used. Of these, genes derived from bacteria belonging to the genus Escherichia are preferred.

As the gene encoding PEP carboxykinase of Escherichia coli (EC number 4.1.1.49), the pckA gene has been elucidated (nucleotide numbers 3530456 to 3532078 in the sequence of GenBank accession NC_{—}000913.1, gi: 16131280). Therefore, the pckA gene can be obtained by PCR (polymerase chain reaction; refer to White, T. J. et al., Trends Genet., 5, 185 (1989)) utilizing primers prepared based on the nucleotide sequence of the gene. Genes encoding PEP carboxykinase of other microorganisms can be obtained in a similar manner.

The pckA gene originated from the genus of Escherichia or its equivalents encompasses a DNA which encodes the protein (A) or (B): (A) a protein having the amino acid sequence of SEQ ID NO:2; (B) a protein having the amino acid sequence of SEQ ID NO:2 which includes deletion, substitution, insertion, or addition of one or several amino acids, and which has an activity of PEP carboxykinase.

The DNAs which code for proteins of the present invention include a DNA coding for a protein which includes deletion, substitution, insertion, or addition of one or several amino acids in one or more positions on the protein (A), as long as they do not lose the activity of the protein. Although the number of “several” amino acids referred to herein differs depending on the position or the type of amino acid residues in the three-dimensional structure of the protein, it may be 2 to 50, preferably 2 to 25, and more preferably 2 to 10 for the protein (A). The DNA coding for substantially the same protein as the protein defined in (A) may be obtained by, for example, modification of the nucleotide sequence coding for the protein defined in (A). For example, site-directed mutagenesis can be employed so that deletion, substitution, insertion, or addition of an amino acid residue or residues occurs at a specific site. Such modified DNA can be obtained by conventional methods, such as by treating with reagents or other conditions generating mutations. Such methods include treatment of the DNA coding for proteins of the present invention with hydroxylamine, or treatment of the bacterium harboring the DNA with UV irradiation or a reagent such as N-methyl-N′-nitro-N-nitrosoguanidine or nitrous acid.

The DNA coding for the proteins of the present invention include variants which can be found in the different strains and variants of bacteria belonging to the genus Escherichia according to natural diversity. The DNA coding for such variants can be obtained by isolating the DNA, which hybridizes with pckA gene or a part of the gene under stringent conditions, and which encodes the protein having the activity of PEP carboxykinase. The term “stringent conditions” referred to herein include conditions under which a so-called specific hybrid is formed, and a non-specific hybrid is not formed. For example, the stringent conditions include a condition under which DNAs having high homology, for instance DNAs having homology of 70% or more, preferably 80% or more, more preferably 90% or more, and most preferably 95% or more, are hybridized with each other. Alternatively, stringent conditions can include conditions which comprise ordinary washing conditions for Southern hybridization, e.g., 60° C., 1×SSC, 0.1% SDS, preferably 0.1×SSC, 0. 1% SDS. As a probe for the DNA that codes for variants and hybridizes with pckA gene, a partial sequence of the nucleotide sequence of SEQ ID NO: 1 can also be used. Such a probe may be prepared by PCR using oligonucleotides produced based on the nucleotide sequence of SEQ ID NO: 1 as primers, and a DNA fragment containing the nucleotide sequence of SEQ ID NO: 1 as a template. When a DNA fragment of about 300 bp in length is used as the probe, the washing conditions of the hybridization can be, for example, 50° C., 2×SSC, and 0.1% SDS.

The activity of PEP carboxykinase can be enhanced by, for example, transforming the bacterium belonging to the genus Escherichia with a recombinant DNA, which has been prepared by ligating a gene fragment encoding PEP carboxykinase with a vector that functions in the bacterium belonging to the genus Escherichia, preferably a multi-copy type vector. The activity of PEP carboxylase is enhanced as a result of the increase in copy numbers of the genes encoding the enzymes in the transformant strain.

Transformation of a bacterium with a DNA coding for a protein means introduction of the DNA into a bacterium, for example, by conventional methods to increase expression of the gene coding for the protein of present invention and to enhance the activity of the protein in the bacterial cell. The activity of PEP carboxykinase can also be enhanced by enhancing transcription and/or translation of a gene encoding PEP carboxykinase. Thus the bacterium of the present invention includes one wherein the activity of the protein of the present invention is enhanced by alteration of an expression control sequence of the DNA coding for the protein as defined in (A) or (B) on the chromosome of the bacterium. The enhancement of gene expression can be achieved by locating the DNA of the present invention under the control of a more potent promoter in place of the native promoter. The term “native promoter” means a DNA region present in a wild-type organism which is located upstream of the open reading frame (ORF) of the gene and has the function of promoting expression of the gene. The sequence of the native promoter of the pckA gene was presented by Ramseier T. M. et al. (Mol. Microbiol., 1995, 16, 6, 1157-1169) and is available in EMBL/GenBank database under accession number U21325. The strength of a promoter is defined by the frequency of RNA synthesis initiation acts. A method for evaluating the strength of a promoter is described by, for example, Deuschle U., Kammerer W., Gentz R., Bujard H. (Promoters in Escherichia coli: a hierarchy of in vivo strength indicates alternate structures. EMBO J. 1986, 5, 2987-2994).

The FruR protein (also known as Cra—Catabolite Repressor/Activator) is a global regulatory protein that modulates the direction of carbon flow. Key enzymes of most central carbohydrate metabolic pathways are the targets of a cAMP-independent mechanism of catabolite repression in enteric bacteria mediated by the FruR protein. In the presence of appropriate exogenous sugar substrates, expression of genes encoding enzymes of the glycolytic and Entner-Doudoroff pathways, including some genes encoding key proteins of the PTS, is activated. At the same time, expression of genes encoding key enzymes of gluconeogenic, glyoxalate shunt, and Krebs cycle pathways is inhibited. The FruR protein regulates expression of all of these genes. The FruR represses synthesis of key enzymes of the glycolytic and Entner-Doudoroff pathways and activates synthesis of key enzymes of the three other pathways (Escherichia coli and Salmonella, Second Edition, Editor in Chief: F. C. Neidhardt, ASM Press, Washington D.C., 1996). PEP carboxykinase encoded by pckA gene is the gluconeogenic enzyme. Since it is known that the PppsA promoter is up to 20 times more potent for the FruR protein while the PpckA promoter is only potent for FruR up to 4 times (Saier M. H., Jr., and Ramseier T. M., J. Bacteriol., 1996, 178, 12, 3411-3417), the PppsA promoter could be assumed to be a more potent promoter then PpckA promoter in the strains constitutively expressing the fruR gene.

The enhancement of translation can be achieved by introducing into the DNA of the present invention a more efficient Shine-Dalgarno sequence (SD sequence) in the place of the native SD sequence. The SD sequence is a region upstream of the start codon of the mRNA which interacts with the 16S RNA of ribosome (Shine J. and Dalgarno L., Proc. Natl. Acad. Sci. U.S.A., 1974, 71, 4, 1342-6). The term “native SD sequence” means SD sequence present in the wild-type organism. The nucleotide sequence of the native SD sequence of the pckA gene, which is a part of promoter region, was presented by Ramseier T. M. et al. (Mol. Microbiol., 1995, 16, 6, 1157-1169) and is available in EMBL/GenBank database under accession number U21325. The SD sequence of the φ10 gene from phage T7 is an example as an efficient SD sequence (Olins P. O. et al., Gene, 1988, 73, 227-235).

Methods for preparation of chromosomal DNA, hybridization, PCR, preparation of plasmid DNA, digestion and ligation of DNA, transformation, selection of an oligonucleotide as a primer, and the like may be ordinary methods well known to one skilled in the art. These methods are described in, for example, Sambrook, J., and Russell D., “Molecular Cloning A Laboratory Manual, Third Edition”, Cold Spring Harbor Laboratory Press (2001) and the like.

The bacterium of the present invention can be obtained by introduction of the aforementioned DNAs into a bacterium inherently having the ability to produce an L-amino acid. Alternatively, the bacterium of present invention can be obtained by imparting the ability to produce L-amino acid to a bacterium already harboring the DNAs.

As a parent strain which has enhanced activity of the protein of the present invention, an L-tryptophan-producing bacterium belonging to the genus Escherichia such as the E. coli strains JP4735/pMU3028 (DSM10122) and JP6015/pMU91 (DSM10123) which is deficient in the tryptophanyl-tRNA synthetase encoded by mutant trpS gene (U.S. Pat. No. 5,756,345); E. coli strain SV164 (pGH5) having serA allele which is free from feedback inhibition by serine (U.S. Pat. No. 6,180,373); E. coli strains AGX17 (pGX44) (NRRL B-12263) and AGX6(pGX50)aroP (NRRL B-12264) deficient in the enzyme tryptophanase (U.S. Pat. No. 4,371,614); E. coli strain AGX17/pGX50,pACKG4-pps in which a phosphoenolpyruvate-producing ability is enhanced (WO9708333, U.S. Pat. No. 6,319,696), and the like, may be used.

Previously, the authors of the present invention identified that when the wild-type allele of the yddG gene which encodes a membrane protein, not involved in a biosynthetic pathway of any L-amino acid, was amplified on a multi-copy vector in a microorganism, resistance to L-phenylalanine and several other amino acid analogues was imparted to the microorganism. Besides, the yddG gene can enhance production of L-phenylalanine or L-tryptophan when additional copies of the gene are introduced into the cells of the respective producing strain (WO03044192). The L-tryptophan-producing bacterium can be further modified to have enhanced expression of the yddG open reading frame or genes effective for L-tryptophan biosynthesis, which include genes of trpEDCBA operon, genes of the common pathway for aromatic acids such as aroF, aroG, aroH, aroB, aroD, aroE, aroK, aroL, aroA, and aroC genes, genes of L-serine biosynthesis such as serA, serB, and serC genes, and the like.

The method of present invention includes a method for producing an L-amino acid comprising cultivating the bacterium of the present invention in a culture medium, allowing the L-amino acid to be produced and accumulated in the culture medium, and collecting the L-amino acid from the culture medium. Also the method of the present invention includes a method for producing L-tryptophan which includes cultivating the bacterium of the present invention in a culture medium, allowing L-tryptophan to be produced and accumulated in the culture medium, and collecting L-tryptophan from the culture medium.

In the present invention, cultivation, collection, and purification of L-amino acids such as L-tryptophan from the medium and the like may be performed in a manner similar to a conventional fermentation method wherein an amino acid is produced using a microorganism. A medium used for culture may be either a synthetic or a natural medium, so long as the medium contains a carbon source and a nitrogen source and minerals and, if necessary, appropriate amounts of nutrients which the microorganism might require for growth. The carbon source may include various carbohydrates such as glucose and sucrose, and various organic acids. Depending on the mode of assimilation of the chosen microorganism, alcohol, including ethanol, and glycerol may be used. As a nitrogen source, various ammonium salts such as ammonia and ammonium sulfate, other nitrogen compounds such as amines, a natural nitrogen source such as peptone, soybean-hydrolysate, and a digested fermentative microorganism can be used. As minerals, potassium monophosphate, magnesium sulfate, sodium chloride, ferrous sulfate, manganese sulfate, calcium chloride, and the like, are used. Additional nutrients can be added to the medium, if necessary. For instance, if the microorganism requires tyrosine for growth (tyrosine auxotrophy), a sufficient amount of tyrosine can be added to the cultivation medium.

The cultivation is performed preferably under aerobic conditions such as by shaking and/or stirring with aeration, at a temperature of between 20 to 42° C., preferably between 37 to 40° C. The pH of the culture is usually between 5 and 9, preferably between 6.5 and 7.2. The pH of the culture can be adjusted with ammonia, calcium carbonate, various acids, various bases, and buffers. Usually, a 1 to 5-day cultivation leads to the accumulation of the target L-amino acid in the liquid medium.

After cultivation, solids such as cells can be removed from the liquid medium by centrifugation or membrane filtration, and the target L-amino acid can be subsequently collected and purified by ion-exchange, concentration, and/or crystallization methods.

### EXAMPLES

The present invention will be more concretely explained below with reference to the following non-limiting Examples. In the Examples, an amino acid is of L-configuration unless otherwise noted.

### Example 1

### Construction of the Plasmid Carrying the fruR Gene and the Mutant serA Gene Encoding for a Protein Which is Not Subject to Feedback Inhibition by Serine

An L-tryptophan producing-strain SV164(pGH5) (U.S. Pat. No. 6,180,373) contains the pGH5 plasmid which carries a mutant serA5 gene coding for a protein which is not subject to feedback inhibition by serine. Amplification of the serA5 gene is necessary for increasing the amount of serine, which is the precursor of L-tryptophan.

On the other hand, it is also known that P_{ppsA} promoter is activated by the FruR protein up to 20 times while P_{pckA} promoter is activated by FruR only 4 times (Saier M. H., Jr., and Ramseier T. M., J. Bacteriol., 1996, 178, 12, 3411-3417). Since the object is to enhance activity of PEP carboxykinase by substituting the P_{pckA} promoter with the P_{ppsA} promoter, it was necessary to express the fruR gene constitutively.

For accomplishing the above two goals, the plasmid pGH5 in the L-tryptophan-producing strain was substituted with pMW-P_{lacUV5}-serA5-fruR plasmid, which was constructed by the following scheme.

Based on the known nucleotide sequence of pGH5 plasmid (U.S. Pat. No. 6,180,373) the primers depicted in SEQ ID No.3 (primer A) and No. 4 (primer B) were synthesized. Primer A is complementary to a sequence at the start codon region of the serA5 gene which has a restriction enzyme XbaI recognition site which has been introduced into the 5′-end thereof. Primer B is complementary to a sequence of the termination codon region of serA5 gene which has a restriction enzyme SalI recognition site which has been introduced into the 5′-end thereof.

The pGH5 plasmid was isolated by ordinary methods. PCR was carried out on “ThermoHybaid PCRExpress PCR system” under the following conditions: 30 sec at 95° C., 30 sec at 55° C., 30 sec at 72° C., 25 cycles with a Taq polymerase (MBI Fermentas).

Concurrently, the fruR gene was amplified. The chromosomal DNA of E. coli strain W 3350 was isolated by ordinary methods. Primers C (SEQ ID No. 5) and D (SEQ ID No. 6) were used. Primer C (complementary to a start codon region of fruR gene) contains a SalI-restriction site. Primer D (complementary to a stop codon region of fruR gene) contains a HindIII-restriction site. PCR was conducted as follows: 30 sec at 95° C., 30 sec at 53° C., 30 sec at 72° C., 25 cycles with a Taq polymerase (MBI Fermentas).

The PCR fragments containing the serA5 and fruR genes obtained as described above were treated with SalI and then ligated. The ligated product was digested by XbaI and HindIII, and inserted into the pMW-P_{lacUV5}-lacZ plasmid (Mashko S. V., et al., Biotekhnologiya (rus), 2001, 5, 3-20), which had been previously treated with the same restriction enzymes. Thus the pMW-P_{lacUV5}-serA5-fruR plasmid was obtained.

### Example 2

### Cloning the yddG Gene from E. coli

The yddG gene encodes a transmembrane protein useful for L-tryptophan production (WO03/044192), and was cloned using the primers depicted in SEQ ID No. 7 (primer yddg1) and No. 8 (primer yddg2). Primer yddg1 is complementary to a sequence from 91 to 114 bp downstream of the termination codon of the yddG gene, and has a restriction enzyme BamHI recognition site which has been introduced into the 5′-end thereof. Primer yddg2 is complementary to a sequence from 224 to 200 bp upstream of the start codon of the yddG gene, and has a restriction enzyme SalI recognition site which has been introduced into the 5′-end thereof.

The chromosomal DNA of E. coli strain TG1 was prepared by ordinary methods. PCR was carried out using “Perkin Elmer GeneAmp PCR System 2400” under the following conditions: 40 sec. at 95° C., 40 sec. at 47° C., 40 sec. at 72° C., 30 cycles with a Taq polymerase (Fermentas). The obtained PCR fragment containing the yddG gene with its own promoter was treated with BamHI and SalI, and inserted into the multicopy vector pAYCTER3, which had been previously treated with the same enzymes. Thus, the plasmid pYDDG2 was obtained.

The pAYCTER3 vector is a derivative of pAYC32. pAYC32 is a moderate copy-number vector and very stable, and is constructed on the basis of plasmid RSF 1010 which contains a marker for streptomycin resistance (Christoserdov A. Y., Tsygankov Y. D, Broad-host range vectors derived from a RSF 1010 Tn1 plasmid, Plasmid, 1986, v. 16, pp. 161 -167). The pAYCTER3 vector was obtained by introducing the polylinker from the pUC19 plasmid and the strong terminator rrnB into the pAYC32 plasmid in place of its promoter as follows. At first, the polylinker from pUC19 plasmid was obtained by PCR using the primers depicted in SEQ ID No. 9 and No. 10. The obtained PCR product was treated with EcoRI and BglII restriction enzymes. The terminator rrnB was also obtained by PCR using the primers depicted in SEQ ID No. 11 and No. 12. The obtained PCR product was treated with BglII and BclI restriction enzymes. Then, these two DNA fragments were ligated into pAYC32 plasmid, which had been previously treated with EcoRI and BclI restriction enzymes. Thus the pAYCTER3 plasmid was obtained.

### Example 3

### Substitution of the Native Upstream Region of the pckA Gene with the Hybrid Regulatory Element Carrying the P_{ppsA} Promoter and SD_{φ10} in the E. coli Chromosome

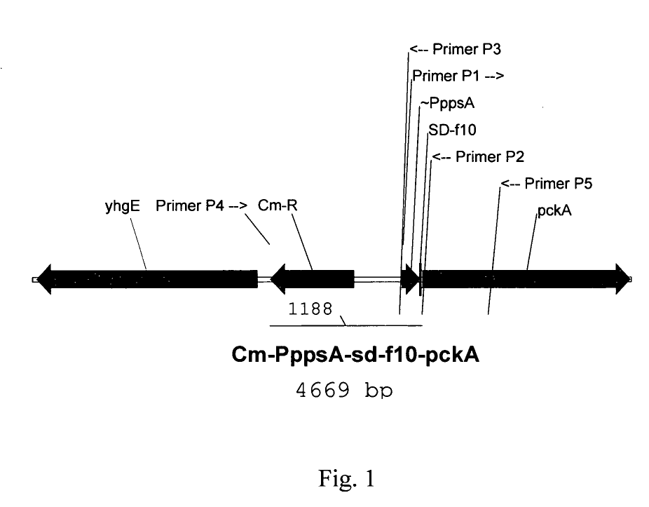

To enhance the pckA gene expression, the promoter region of the ppsA gene (P_{ppsA}) derived from the E. coli chromosome was linked to the Shine-Dalgarno sequence (SD sequence) of the φ10 gene from phage T7. This fragment was integrated upstream of the pckA coding region into the chromosome of the E. coli strain BW25113(pKD46) in place of the native region using a method described by Datsenko K. A. and Wanner B. L. (Proc. Natl. Acad. Sci. USA, 2000, 97, 6640-6645), which is also called “Red-mediated integration”. In addition, the artificial DNA fragment integrated into the corresponding region of the bacterial chromosome carried the chloramphenicol resistance gene (Cm^{R}) (FIG. 1). The nucleotide sequence of the substituted native region located upstream of pckA gene is presented in the Sequence listing (SEQ ID NO: 1). Escherichia coli strain BW25113 containing the recombination plasmid pKD46 can be obtained from the E. coli Genetic Stock Centre, Yale University, New Haven, USA, the accession number of which is CGSC7630 (WO02103010A1).

Construction of the above-mentioned artificial DNA fragment was accomplished by the following steps. First, a DNA fragment was obtained by PCR having the BglII—restriction site in the “upstream” region, the P_{ppsA} promoter, and the SD sequence from the phage T7 φ10 gene linked directly to the ATG-initiation codon of the pckA gene in the “downstream” region. The chromosomal DNA from E. coli W3350 strain was used as the template in PCR. PCR was conducted using primers P1 (SEQ ID NO: 13) and P2 (SEQ ID NO: 14). Primer P1 contains a BglII—restriction site. Primer P2 contains the SD sequence of the phage T7 φ10 gene as well as 36 nucleotides from the pckA open reading frame. The sequence from the pckA gene was introduced into primer P2 for further Red-mediated integration of the fragment into the bacterial chromosome.

In all cases, PCR was conducted using the amplificatory “ThermoHybaid PCR Express PCR System”. The reaction mixture having a total volume of 50 μl contains 5 μl of 10× PCR-buffer with 15 mM MgCl₂ (“Fermentas”, Lithuania), 200 μM each of dNTP, 20 nM each of the exploited primers and 1 u Taq-polymerase (“Fermentas”, Lithuania). 0.5 μg of the chromosomal DNA was added to the reaction mixture as the template DNA for further PCR-driven amplification. The PCR conditions were as follows: initial DNA denaturation for 5 min at 95° C., followed by 25 cycles of denaturation at +95° C. for 30 sec, annealing at +55° C. for 30 sec, elongation at +72° C. for 30 sec, and the final polymerization for 7 min at +72° C.

Concurrently, the second stage of construction of the DNA fragment of interest was conducted. The Cm^{R} gene was amplified by PCR using the commercially available plasmid pACYC184 (GenBank/EMBL accession number X06403, “Fermentas”, Lithuania) as the template, and primers P3 (SEQ ID NO: 15) and P4 (SEQ ID NO: 16). Primer P3 contains the BglII-restriction site used for joining the previously obtained DNA fragment containing the P_{ppsA} promoter. Primer P4 contains 36 nucleotides which are located upstream of the pckA gene derived from E. coli and are necessary for further Red-mediated integration of the fragment into the bacterial chromosome.

Amplified DNA fragments were then concentrated by agarose gel-electrophoresis and extracted from the gel by centrifugation through “GenElute Spin Columns” (“Sigma”, USA), and followed by ethanol precipitation.

The two obtained DNA fragments were treated with BglII restriction endonuclease followed by ligation using T4 DNA ligase (Maniatis T., Fritsch E. F., Sambrook, J.: Molecular Cloning: A Laboratory Manual. 2^{nd} edn. Cold Spring Harbor, N.Y.: Cold Spring Harbor Laboratory Press, 1989).

The ligated product was amplified by PCR using primers P2 and P4. PCR was conducted in a reaction mixture having a total volume of 50 μl and containing 5 μl of 10× AccuTaq LA buffer (“Sigma”, USA), 200 μM each of dNTP, 20 nM each of the exploited primers and 1 μ AccuTaq LA polymerase (“Sigma”, USA). Approximately 50 ng of the ligated DNA products was added to the reaction mixture as the template DNA. The PCR conditions were as follows: initial DNA denaturation for 5 min at 95° C. followed by 25 cycles of denaturation at 95° C. for 30 sec, annealing at 55° C. for 30 sec, elongation at 72° C. for 4 min and the final polymerization for 7 min at 72° C.

The structure of constructed DNA region upstream of the pckA gene is shown in FIG. 1. The nucleotide sequence of the constructed DNA region is presented in SEQ ID NO: 18.

The DNA fragment obtained and purified as described above was used for electroporation and Red-mediated integration, into the bacterial chromosome of the E. coli strain BW25113(pKD46). The recombinant plasmid pKD46 (Datsenko, K. A., Wanner, B. L., Proc. Natl. Acad. Sci. USA, 2000, 97, 6640-6645) having the thermosensitive replicon was used as the donor of the phage λ-derived genes responsible for the Red-mediated recombination system.

The cells of BW25113(pKD46) were grown overnight at 30° C. in a liquid LB-medium also containing ampicillin (100 μg/ml), then diluted 1:100 with the SOB-medium (Yeast extract, 5 g/l; NaCl, 0.5 g/l; Tryptone, 20 g/l; KCl, 2.5 mM; MgCl₂, 10 mM) containing ampicillin (100 μg/ml) and L-arabinose (10 mM) (arabinose is used for inducing the plasmid encoding genes of the Red system) and grown at 30° C. until reaching the optical density OD₆₀₀=0.4-0.7 of the bacterial culture. The grown cells from 10 ml of the bacterial culture were washed 3 times by ice-cold de-ionized water followed by suspension in 100 μl of water. 10 μl of DNA fragment (100 ng) dissolved in de-ionized water was added to the cell suspension. The electroporation was performed by “Bio-Rad” electroporator (USA) (No. 165-2098, version 2-89) according to the manufacturer's instructions. Shocked cells were added to 1-ml of SOC medium (Sambrook et al., “Molecular Cloning A Laboratory Manual, Second Edition”, Cold Spring Harbor Laboratory Press (1989)), incubated for 2 hours at 37° C., and then spread onto L-agar containing 25 μg/ml of chloramphenicol. Colonies which grew within 24 h were tested for the presence of Cm^{R} marker upstream of the pckA gene by PCR using primers P4 (SEQ ID NO: 16) and P5 (SEQ ID NO: 17). The same colonies were also tested for the presence of the P_{ppsA} promoter region upstream of pckA gene by PCR using primers P1 (SEQ ID NO: 13) and P5 (SEQ ID NO: 17). For these tests, a freshly isolated colony was suspended in 20 μl water, of which 1 μl was used in PCR. PCR conditions were as follows: initial DNA denaturation for 10 min at 95° C.; then 30 cycles of denaturation at 95° C. for 30 sec, annealing at 55° C. for 30 sec and elongation at 72° C. for 1 min; the final polymerization for 7 min at 72° C. A few of the Cm^{R} colonies tested contained the desired 1749 nt and 697 nt DNA fragments, which confirmed the presence of the entire constructed DNA region upstream of the pckA gene and the hybrid regulatory element, carrying the P_{ppsA} promoter and SD_{φ10} in E. coli chromosome, respectively. One of the obtained strains was cured from the thermosensitive plasmid pKD46 by culturing at 37° C. The resulting strain was named E. coli strain BW25113 (Cm-P_{ppsA}-SD_{φ10}-pckA).

### Example 4

### Effect of Enhanced pckA Gene Expression on Tryptophan Production

The tryptophan-producing E. coli strain SV164 (pMW-P_{lacUV5}-serA5-fruR, pYDDG2) was used as a parental strain for evaluating the effect of enhanced pckA gene expression on tryptophan production. The strain SV164 is described in detail in U.S. Pat. No. 6,180,373.

To test the effect of enhancing the pckA gene expression which has been placed under the control of the P_{ppsA} promoter and SD of phage T7 φ10 gene on tryptophan production, the above-mentioned DNA fragment from the chromosome of E. coli strain BW25113 (Cm-P_{ppsA}-SD_{φ10}-pckA) was transferred to the tryptophan-producing E. coli strain SV164 (pMW-P_{lacUV5}-serA5-fruR) by P1 transduction (Miller, J. H. (1972) Experiments in Molecular Genetics, Cold Spring Harbor Lab. Press, Plainview, N.Y.). Then, plasmid pYDDG2 was introduced into both the SV164 (pMW-P_{lacUV5}-serA5-fruR) strain and the resulting transductant.

Both the SV164 (pMW-P_{lacUV5}-serA5-fruR, pYDDG2) and the SV164 P_{ppsA}-SD_{φ10}-pckA (pMW-P_{lacUV5}-serA5-fruR, pYDDG2) strains were cultivated overnight with shaking at 37° C. in 3 ml of nutrient broth supplemented with 30 μg/ml of ampicillin and 25 μg/ml of streptomycin. 0.3 ml of the obtained cultures were inoculated into 3 ml of a fermentation medium containing tetracycline (20 μg/ml) in 20×200 mm test tubes, and cultivated at 37° C. for 48 hours on a rotary shaker at 250 rpm.

The composition of the fermentation medium is presented in Table 1.

TABLE 1

| Sections | Component | Final concentration, g/l |
|---|---|---|
| A | KH₂PO₄ | 1.5 |
| | NaCl | 0.5 |
| | (NH₄)₂SO₄ | 1.5 |
| | L-Methionine | 0.05 |
| | L-Phenylalanine | 0.1 |
| | L-Tyrosine | 0.1 |
| | Mameno (total N) | 0,07 |
| B | Glucose | 40.0 |
| | MgSO₄ .7H₂O | 0.3 |
| C | CaCl₂ | 0.011 |
| D | FeSO₄ .7H₂O | 0.075 |
| | Sodium citrate | 1.0 |
| E | Na₂MoO₄ .2H₂O | 0.00015 |
| | H₃BO₃ | 0.0025 |
| | CoCl₂ .6H₂O | 0.00007 |
| | CuSO₄ .5H₂O | 0.00025 |
| | MnCl₂ .4H₂O | 0.0016 |
| | ZnSO₄ .7H₂O | 0.0003 |
| F | Thiamine HCl | 0.005 |
| G | CaCO₃ | 30.0 |
| H | Pyridoxine | 0.03 |

After cultivation, the amount of L-tryptophan which accumulated in the medium was determined by TLC. 10×15 cm TLC plates coated with 0.11 mm layers of Sorbfil silica gel without fluorescent indicator (Stock Company Sorbpolymer, Krasnodar, Russia) were used. Sorbfil plates were developed with a mobile phase: propan-2-ol : ethylacetate: 25% aqueous ammonia: water=16:16:3:9 (v/v). A 2% solution of ninhydrin in acetone was used as a visualizing reagent.

The results are presented in the Table 2.

TABLE 2

| | | Amount of |
|---|---|---|
| E. coli strain | OD₆₀₀ | tryptophan, g/l |
| SV164 (pMW-P_{lacUV5}-serA5-fruR, pYDDG2) | 7.5 | 4.20 |
| SV164 P_{ppsA}-SD_{φ10}-pckA | 7.5 | 4.61 |
| (pMW-P_{lacUV5}-serA5-fruR, pYDDG2) | | |

As it can be seen from the Table 2, enhancing pckA gene expression improved tryptophan productivity of the SV164 (pMW-P_{lacUV5}-serA5-fruR) strain.

While the invention has been described with reference to preferred embodiments thereof, it will be apparent to one skilled in the art that various changes can be made, and equivalents employed, without departing from the scope of the invention. All the cited references herein, including the foreign priority document, RU 2003109477, are incorporated as a part of this application by reference in its entirety.

### INDUSTRIAL APPLICABILITY

According to the present invention, productivity of an aromatic L-amino acid-producing strain can be enhanced and a method for producing the aromatic L-amino acid using the strain is provided. The present invention is useful in the fermentation industry.

1. An L-amino acid-producing bacterium belonging to the genus Escherichia, wherein the bacterium has been modified to have enhanced PEP carboxykinase activity.

2. The bacterium according to claim 1, wherein the PEP carboxykinase activity is enhanced by modifying an expression control sequence of the PEP carboxykinase gene on the chromosome of the bacterium so that the expression of the gene is enhanced, or by increasing the copy number of the gene.

3. The bacterium according to claim 2, wherein a native promoter of said gene is replaced with a more potent promoter.

4. The bacterium according to claim 2, wherein a native SD sequence of said gene is replaced with a more efficient SD sequence.

5. The bacterium according to claim 2, wherein the PEP carboxykinase gene is originated from a bacterium belonging to the genus Escherichia.

6. The bacterium according to claim 5, wherein the PEP carboxykinase gene encodes a protein selected from the group consisting of:
(A) a protein comprising the amino acid sequence in SEQ ID NO: 2; and
(B) a protein comprising the amino acid sequence in SEQ ID NO: 2 which includes deletion, substitution, insertion, or addition of one or several amino acids, and which has a PEP carboxykinase activity.

7. The bacterium according to claim 5, wherein the PEP carboxykinase gene comprises a DNA selected from the group consisting of:
(a) a DNA comprising the nucleotide sequence of the nucleotides 1 to 1623 in SEQ ID NO: 1; and
(b) a DNA which is able to hybrid with the nucleotide sequence of the nucleotides 1-1623 in SEQ ID NO:1, or is able to hybridize with a probe which can be prepared from said nucleotide sequence under stringent conditions and encodes a protein having PEP carboxykinase activity.

8. The bacterium according to claim 7, wherein the stringent conditions are conditions in which washing is performed at 60° C., and at a salt concentration corresponding to 1×SSC and 0.1% SDS.

9. The bacterium according to claim 1, wherein the bacterium is further modified to have enhanced expression of a yddG open reading frame.

10. The bacterium according to claim 1, wherein the L-amino acid comprises an aromatic L-amino acid selected from the group consisting of L-tryptophan, L-phenylalanine and L-tyrosine.

11. A method for producing an aromatic L-amino acid comprising cultivating the bacterium according to claim 1 in a culture medium, and collecting the L-amino acid from the culture medium.

12. The method according to claim 11, wherein the L-amino acid comprises an aromatic amino acid selected from the group consisting of L-tryptophan, L-phenylalanine, and L-tyrosine.

13. The method according to claim 12, wherein the bacterium has enhanced expression of genes for aromatic amino acid biosynthesis.
